(19) United States
(12) Patent Application Publication
(10) Pub. No.: US 2025/0228779 A1
(43) Pub. Date: Jul. 17, 2025

(54) METHOD AND COMPOSITION FOR TREATING PULMONARY FIBROSIS

(71) Applicant: MannKind Corporation, Danbury, CT (US)

(72) Inventors: John J. Freeman, JR., New Fairfield, CT (US); Michael Castagna, Danbury, CT (US)

(21) Appl. No.: 19/169,921

(22) Filed: Apr. 3, 2025

Related U.S. Application Data
(63) Continuation of application No. 18/265,660, filed on Jun. 6, 2023.
(60) Provisional application No. 63/122,289, filed on Dec. 7, 2020.

(51) Int. Cl. A61K 9/16; A61K 9/00; A61K 31/4412; A61K 31/496

(52) U.S. Cl. CPC A61K 9/1617; A61K 9/0075; A61K 9/1623; A61K 31/4412; A61K 31/496

(57) ABSTRACT

A method, composition and kit for the treatment of interstitial lung disease are disclosed. The method utilizes a combination product for inhalation comprising a therapeutic amount of a dry powder formulation provided in an inhaler to be administered to a subject in need by oral inhalation. The composition comprise diketopiperazine particles for pulmonary inhalation.

### TECHNICAL FIELD

The present disclosure relates to methods, compositions and kits for therapeutic treatment of idiopathic pulmonary fibrosis. In particular, the methods, compositions and kits comprise a combination product comprising a dry powder and an inhaler, which dry powder is for administration to a patient by oral inhalation.

### BACKGROUND

Idiopathic pulmonary fibrosis (IPF) is a chronic lung disease of yet unknown causes and there is no cure for IPF. The disease is progressive and irreversible and causes scar tissue (fibrosis) to build up in the lungs, which makes the lungs unable to transport oxygen into the bloodstream effectively. It affects people between the ages of 50 and 70. It belongs to a group of conditions called interstitial lung diseases (ILD), which describes lung diseases that involve inflammation or scarring in the lung. The most common signs and symptoms of IPF are shortness of breath and a persistent dry, hacking cough. Subjects affected with IPF also experience a loss of appetite and gradual weight loss. In individuals with IPF, scarring of the lungs increases over time until the lungs can no longer provide enough oxygen to the body's organs and tissues.

Currently, there are no procedures, or medications that can remove the progressive scarring of lung tissue. Therefore, it is important to learn good coping skills and educate the patient about the disease. Generally, treatments are designed to slow progression of scar formation in the lungs and these may not necessarily lessen the symptoms of cough and breathlessness associated with the disease. Oral tablet of pirfenidone and nintedanib therapies have been shown to slow the progression of IPF; however, some patients cannot tolerate these medications at the dosage needed to slow down progression due to the side effects. With repeated and necessary high dosing to slow disease progression, there are too many adverse effects, including, gastrointestinal such as nausea, diarrhea, abdominal pain, vomiting; hepatobiliary, nervous system, vascular, metabolism and nutritional disorders.

There are some additional medications that are useful to improve the symptoms of IPF, including, shortness of breath and cough. Some of these medications include, for example, anti-acids to prevent gastroesophageal reflux and opioids to treat the shortness of breath. Oxygen therapy and exercise training to increase oxygen levels are recommended to subjects with IPF, as well as education and support for people with chronic condition in order to provide them with pulmonary rehabilitation. Moreover, one major and invasive treatment is to provide the patient with lung transplant. Therefore, there is a need to improve or provide a patient with IPF alternate and new methods of treatment to treat the disease.

Drug delivery to lung tissue is accomplished using a variety of methods and routes of administration. For example, oral drug delivery, or enterally, such as tablets and capsules containing the medication, and parenterally, including, injections of targeted drugs to treat the disease or symptoms of the disease. Devices for inhalation, including, nebulizers and inhalers, such as metered dose inhalers and dry powder inhalers to treat local respiratory tract or lung disease or disorders are also used.

Some dry powder inhaler products developed for pulmonary delivery have met with success to date. However, due to lack of practicality for use, and/or cost of manufacture, there is room for improvement. Some of the persistent problems observed with prior art inhalers, include, lack of device ruggedness, inconsistency in dosing, inconvenience of the equipment, and poor deagglomeration of the powders. With some devices, the need to use harmful propellants to deliver a dose has limited therapy, and high manufacturing costs, and/or lack of patient compliance discourages their production. Therefore, the inventors have identified the need to design and manufacture new formulations and inhalers, which will provide consistent, or improved powder delivery properties, are easy to use, and have discrete configurations which would allow for better patient compliance.

### SUMMARY

Disclosed herein are compositions and methods for using the compositions in the treatment of interstitial lung disease, including idiopathic pulmonary fibrosis. In embodiments herewith, a composition is provided in a dry powder inhaler comprising a replaceable cartridge or capsule comprising a dry powder pharmaceutical formulation for inhalation for delivery to the lungs for local or systemic delivery into the pulmonary circulation. The dry powder inhaler is a breath-powered inhaler which is compact, reusable or disposable, has various shapes and sizes, and comprises a system of airflow conduit pathways for the effective and rapid delivery of powder medicament to the lungs and its systemic circulation.

In a particular embodiment, the method of treating idiopathic pulmonary fibrosis comprises a drug delivery system, which is designed for drug delivery to the lungs by oral inhalation, for rapid delivery and onset of action of the active agent being delivered to lung tissue to reach the alveoli and to systemic circulation in the lungs. In the method, the active agent can reach its target site in a therapeutically effective manner and with less adverse effects. In a particular embodiment, the method of treatment comprises treating or administering to a patient diagnosed with fibrotic and/or inflammatory disease of the lungs, including, idiopathic lung disease, for example, idiopathic pulmonary fibrosis and in need of treatment, a therapeutic dose of a dry powder formulation comprising one or more active agents for treating the disease. In one embodiment, the dose of the dry powder is delivered to the lungs using a dry powder inhaler, and wherein the active agent can reach the deep lung. The pharmaceutical composition is self-administered by the patient with one or more breaths using a breath-powered dry powder inhaler for oral or nasal inhalation. The delivery system can reduce the adverse effects caused by oral tablets or capsule, including, gastrointestinal such as nausea, diarrhea, abdominal pain, vomiting; hepatobiliary, nervous system, vascular, metabolism and nutritional disorders,

In one embodiment, the method further comprises administering to a subject in need of treatment a stable pharmaceutical composition comprising, one or more active agents, for delivery to lung tissue, wherein more than one active agent can be formulated together or formulated separately to be administered separately and at different intervals during a therapy. In a particular embodiment, the pharmaceutical composition comprises a formulation for inhalation comprising a therapeutically effective dose of a dry powder comprising one or more active agents, including, a small molecule such as pirfenidone, pyridone analogs, nintedanib, derivatives thereof, or analogs thereof; and/or combinations thereof. In certain embodiments, the pharmaceutical composition can further comprise any molecule or compound which is suitable for treating idiopathic lung disease and can be present in the composition either alone or in combination with other active agents, including, deoxyribonuclease I (Dnase I) and granulocyte macrophage colony stimulating factors (GM-CSF), anti-inflammatoires, including, kinase inhibitors such as tyrosine kinase inhibitor molecules. The pharmaceutical composition comprises optionally, one or more pharmaceutically acceptable excipients and/or carriers. In this and other embodiments the pharmaceutical composition is provided to the patient in a container, capsule or cartridge for inhalation using a dry powder inhaler.

In one embodiment, an inhalable pharmaceutical composition is disclosed comprising a dry powder comprising a pharmaceutically acceptable excipient, including, a diketopiperazine having the ability for form particles and a therapeutically effective dose of a compound having the formula:

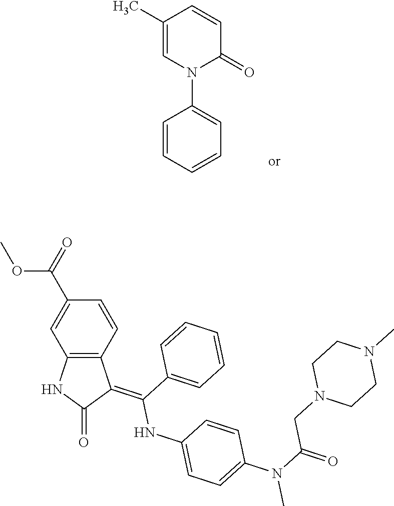

and optionally, one or more pharmaceutically acceptable carriers and/or excipients. In this and other embodiments, the inhalable pharmaceutical composition can be formulated to comprise a dose of one or more active agents for delivering with an inhaler is in an amount of up to 30 mg of an inhalable dry powder per cartridge or capsule, and comprising, optionally, one or more pharmaceutically acceptable salt thereof, including, nintedanib esylate, and a pharmaceutically acceptable carrier and/or excipients. Multiple cartridges can be administered per dosing session depending on the patient's need and up to 300 mg of the active agent per day, which can be administered once or more than once times per day. In some embodiments and depending on the patient's needs, the dosing can further be administered twice, thrice or more times daily.

In one embodiment, the inhalable pharmaceutical composition can comprise one or more pharmaceutically acceptable carrier and/or excipient, which is a surfactant, an amino acid, and/or a phospholipid, or combinations thereof.

In a preferred embodiment, the inhalable pharmaceutical composition for treating ILD, including IPF comprises one or more active agents and a diketopiperazine having the formula:

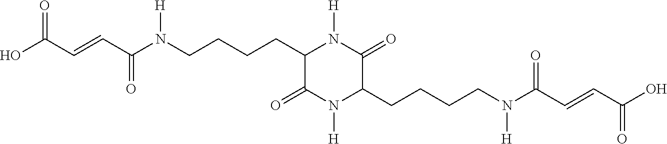 [SMILES: O=C(O)/C=C/C(=O)NCCCCC1NC(=O)C(CCCCNC(=O)/C=C/C(=O)O)NC1=O]

wherein the diketopiperazine is an amorphous powder, in a crystalline form, or in a microcrystalline particle form, or combinations thereof.

In one embodiment, the inhalable pharmaceutical composition is in a crystalline dry powder comprising a therapeutic effective dose of the compound having the formula:

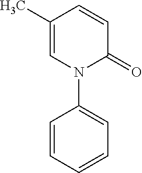

wherein the compound content in a dose of the formulation ranges from about 1 mg to about 50 mg in the dry powder composition.

In some embodiments, the inhalable pharmaceutical composition comprises a dry powder comprising one or more pharmaceutically acceptable carrier and/or excipients selected from lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, microcrystalline cellulose, polyvinylpyrrolidone and polysorbate 80, or combinations thereof.

In other embodiments, the inhalable pharmaceutical composition comprises a dry powder comprising one or more pharmaceutically acceptable carriers and/or excipients selected from the group consisting of sodium citrate, sodium chloride, leucine or isoleucine and trehalose, or combinations thereof.

In certain embodiments, the inhalable pharmaceutical composition comprises microcrystalline particles of 3,6-bis(N-fumaryl-4-aminobutyl)-2,5-diketopiperazine which have a specific surface area ranging from about 20 m²/g to about 63 m²/g. In one embodiment, the microcrystalline particles have a pore size ranging from about 23 nm to about 30 nm.

A method of treating interstitial lung disease, including, idiopathic pulmonary fibrosis comprising, administering to a patient in need of treatment by oral inhalation a dry powder composition comprising diketopiperazine particles and 1 mg to 10 mg; 10 mg to 20 mg; 20 mg to 30 mg, 30 mg to 50 mg; 50 mg to 100 mg; 100 to 150 mg; or 150 to 300 mg per inhalation session of a compound of the formula:

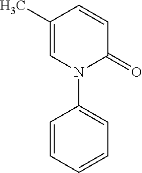

a pharmaceutically acceptable salt thereof, a derivative thereof, and, optionally, a pharmaceutically acceptable carrier and/or excipient, wherein the dry powder composition is provided in a dry powder inhaler in single dose cartridges. In one embodiment, multiple cartridges can be provided to the patient for a predetermined dose depending on the patient's need.

In embodiments herewith, wherein the method comprises pirfenidone, the patient is administered a therapeutically effective dose of the dry powder composition is provided to the patient separately, in a blister, or pouch having one or more capsules or cartridges for adapting to a dry powder inhaler prior use, wherein each capsule or cartridge comprises up to 30 mg, or 50 mg of the compound. In one embodiment, the therapeutically effective dose per day can comprise up 500 mg; up to 750 mg; up to 1,000 mg, or up to 2,500 mg of the compound per day, which is provided in multiple cartridges for inhalation with a dry powder inhaler. The administration can be carried out in one or more dosing sessions.

In this and other aspects, the method utilizes a composition comprising, one or more pharmaceutically acceptable carrier and/or excipients is, which is selected from the group consisting of fumaryl diketopiperazine, lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone, and a surfactant such as polysorbate 80.

In other embodiments, the method for treating interstitial lung disease, including idiopathic pulmonary fibrosis comprises administering to a subject in need of treatment a pharmaceutically effective amount of a dry powder comprising pirfenidone (5-methyl-1-phenyl-2-(1H)-pyridone), or wherein the one or more pharmaceutically acceptable carrier and/or excipient are sodium citrate, sodium chloride, leucine or isoleucine, or trehalose.

In one embodiment, a method of treating pulmonary fibrosis comprises, administering to a patient in need of treatment, an inhalable dry powder pharmaceutical composition comprising a diketopiperazine and a compound having the formula:

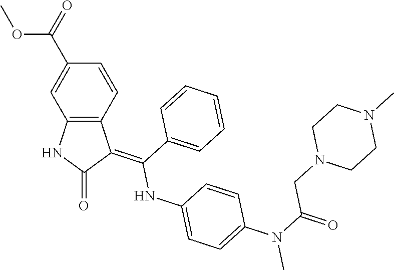

or a pharmaceutically acceptable salt thereof, including, an esylate, and optionally, one or more pharmaceutically acceptable carriers and/or excipients; wherein the diketopiperazine is in an amorphous form, in a crystalline form, or in a crystalline composite particle form, or combinations thereof, and the diketopiperazine has the formula:

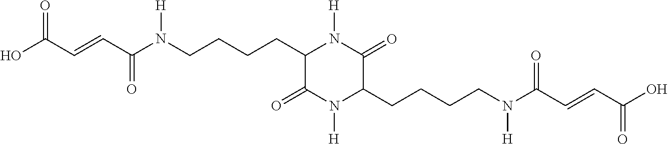 [SMILES: O=C(O)/C=C/C(=O)NCCCCC1NC(=O)C(CCCCNC(=O)/C=C/C(=O)O)NC1=O]

In exemplary embodiments, the method of treating interstitial lung disease and in particular, idiopathic pulmonary fibrosis comprises, administering to a patient in need of treatment and inhalable pharmaceutical dry powder comprising pirfenidone, or nintedanib by oral inhalation using a dry powder inhaler comprising a movable member for mounting a cartridge, or capsule comprising a dose of the dry powder and having a container, which can attain a dosing configuration upon being loaded onto the inhaler, wherein said cartridge comprises the dry powder composition to be inhaled. In one embodiment, the dry powder inhaler cartridge consisting of a lid and a container and a dry powder dose that is provided separately prior to use.

In a particular embodiment, the method of treating IPF comprises providing a patient in need of treatment an inhaler and one or more cartridges comprising a dry powder composition and having the patient inhale the one or more cartridges content from each of the one or more cartridges, wherein the one or more cartridges can deliver an effective dose of up to 300 mg pre dosing session of a compound of the formula:

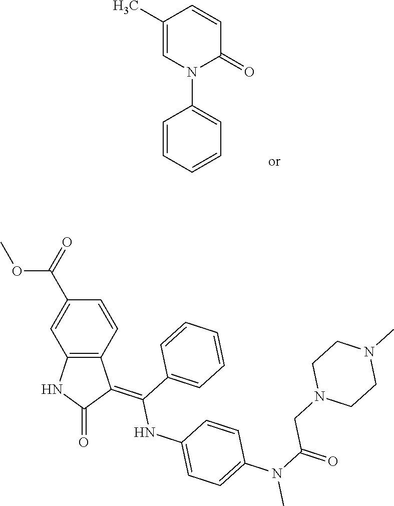

and, wherein the dry powder composition comprises particles of a pharmaceutically acceptable excipient having the formula 3,6-bis(N-fumaryl-4-aminobutyl)-2,5-diketopiperazine. In one embodiment, the method comprises having the patient inhale for at least 4 to 10 seconds, or 2 to 6 seconds per inhalation using a high resistance dry powder inhaler having a resistance value from about 0.05 to about 0.200 (kPa)/liter/min.

In some embodiments, the method of treatment of interstitial lung disease, including, pulmonary fibrosis comprises, administering to a subject in need of treatment, a pharmaceutical composition comprising pirfenidone and/or nintedanib separately, sequentially or combinations thereof with one or more of a vasodilator compound. In one embodiment, the method comprises a combination therapy comprising, administering to the subject a vasodilator, including, sildenafil, tadalafil, vardenafil, a prostaglandin, a prodrug thereof, a prostaglandin derivative, a prostaglandin analog, for example, treprostinil, or a pharmaceutically acceptable salt of these compounds thereof, including, treprostinil sodium, or prodrugs thereof. In a particular embodiment, the method comprises treating interstitial lung disease and pulmonary arterial hypertension simultaneously comprising delivering to the lungs of the patient a combination therapy comprising a dry powder formulation comprising pirfenidone and/or nintedanib and/or a dry powder composition comprising a vasodilator compound, including, treprostinil, treprostinil, or a pharmaceutically acceptable salt of these compounds thereof, including, treprostinil sodium, or prodrugs thereof, and into the systemic circulation of a subject, by pulmonary inhalation using a dry powder inhaler.

In one embodiment, the method comprises providing to a patient in need of treatment a dry powder inhaler comprising the active agent, for example, nintendanib, pirfenidone, or treprostinil in a stable dry powder formulation, and administering the active agent by oral inhalation. In one embodiment, the vasodilator can be formulated together with the pirfenidone, nintedanib in the same formulation or separately and administered separately in its own formulation and provided to the patient at different intervals or sequentially during a dosing session.

In one embodiment, the drug delivery system comprises a dry powder inhaler comprising a diketopiperazine-based drug formulation for delivering small molecules, for example, pirfenidone, nintedanib, a prostaglandin, or analogs thereof, including, tresprostinil and protein-based products for treating pulmonary fibrosis and PAH. The method provides advantages over typical methods of drug delivery, such as, oral tablet and subcutaneous and intravenous injectable/infusion drug products that are sensitive to degradation and/or enzymatic deactivation.

In certain embodiments disclosed herein, a method is provided for the treatment comprises further providing to a patient with pulmonary fibrosis and PAH a prostaglandin, treprostinil, or a pharmaceutically acceptable salt of these compounds thereof, including, treprostinil sodium, or prodrugs thereof or derivative thereof, in a dry powder formulation. The method comprises, selecting a patient to be treated for PAH and interstitial lung disease, and administering to the patient a dry powder formulation comprising, nintedanib, pirfenidone, or treprostinil or a treprostinil salt or derivative thereof; wherein the treprostinil is combined with diketopiperazine microcrystalline particles to produce a pharmaceutical formulation, or composition suitable for pulmonary inhalation and, having the patient inhale from an inhaler containing the composition and delivering the trepostinil formulation using a breath-powered dry powder inhaler. In this and other embodiments, the dry powder formulations is provided in a reconfigurable cartridge comprising from about 1 μg to about 200 μg of treprostinil or a salt thereof in the dry powder formulation per dose. In certain embodiments, the dry powder formulation can comprise from about 10 μg to about 300 μg of treprostinil per dose in a cartridge or capsule. In one embodiment, a cartridge for single use can comprise from about 10 μg to about 90 μg of treprostinil for at least one inhalation. In some embodiments, the dry powder formulation is delivered using at least one inhalation per use. In this and other embodiments, the dry powder formulation is delivered to a patient in less than 10 seconds, or less than 8 seconds or less than 6 seconds per inhalation or breath. In one embodiment, the pharmaceutical dry powder composition comprises microcrystalline particles of fumaryl diketopiperazine wherein the particles have a specific surface area ranging from about 59 m²/g to about 63 m²/g and have a pore size ranging from about 23 nm to about 30 nm.

Also disclosed herein is a method of treating a pulmonary fibrosis concomitant with pulmonary arterial hypertension disease or disorder comprising, selecting a patient to be treated with pulmonary arterial hypertension, or a patient with PAH, which exhibits a condition treatable with an active agent, including treprostinil, epoprostenol, bosentan, ambrisentan, macisentan, sildenafil, tadalafil, riociguat and the like, analogs thereof, or combinations thereof, which patients are treated only by oral or injectable administration, and replacing the aforementioned therapy with an inhalation therapy comprising providing the patient with an inhaler comprising the active agent in a stable dry powder composition for treating the disease or disorder; wherein the stable dry powder composition comprises the active agent and a diketopiperazine; and administering the stable dry powder composition to the patient by pulmonary inhalation; thereby treating the disease or condition.

In an exemplary embodiment, the formulation for treating pulmonary arterial hypertension and/or interstitial lung disease comprises treprostinil or a salt thereof, in an amount up to 200 μg per dose, for example, amounts of 1 μg, 5 μg, 10 μg, 15 μg, 20 μg, 30 μg, 60 μg, 90 μg, 100 μg, 120 μg, 150 μg, 180 μg, 200 μg, or 300 μg, and one or more pharmaceutically acceptable carriers and/or excipients per dose are to be administered to a subject. In this embodiment, the pharmaceutically acceptable carrier and/or excipient can be formulated for oral inhalation and can form particles, for example, a diketopiperazine, including, fumaryl diketopiperazine, sugars such as mannitol, xylitol, sorbitol, and trehalose; amino acids, including, glycine, leucine, isoleucine, methionine; surfactants, including, polysorbate 80; cationic salts, including, monovalent, divalent and trivalent salts, including, sodium chloride, potassium chloride, magnesium chloride, and zinc chloride; buffers such as citrates and tartrates, or combination of one or more carriers and/or excipients and the like. In a particular embodiment, the formulation comprises a dry powder comprising treprostinil, a sugar and an amino acid, wherein the sugar is mannitol or trehalose; and the amino acid is leucine or isoleucine and a cationic salt. In certain embodiments, the formulation can further comprise sodium chloride, potassium chloride, magnesium chloride or zinc chloride, sodium citrate, sodium tartrate, or combinations thereof.

In an exemplary embodiment, a combination therapy comprises a method of treating the interstitial lung disease comprising, administering to a patient a dose of nintedanib, or treprostinil, wherein the nintedanib dose is administered in the same inhaler provided with different cartridges, or from a different inhaler provided with its own cartridges, wherein the treprostinil dose, or the nintedanib dose is administered using a dry powder inhaler for oral inhalation. In this embodiment, a treprostinil inhalation powder dose is provided to a patient suffering with pulmonary arterial hypertension and in need of treatment; wherein the a dry powder inhaler comprises a container including, a cartridge, and the container or cartridge comprises the dry powder comprising treprostinil is administered in multiple daily doses for a period of six months and the treprostinil is administered by oral inhalation at an earlier time in the course of the disease to patients with Functional Class II as a first line monotherapy.

In alternate embodiments, the dry powder for inhalation may be formulated with other carriers and/or excipients other than diketopiperazines, for example a sugar, including trehalose; buffers, including sodium citrate; salts, including, sodium chloride and zinc chloride, and one or more active agents, including, treprostinil, vardenafil, and sildenafil.

In embodiments herewith, the method of treating interstitial lung disease in a patient also with PAH comprises, administering to a patient with moderate to severe PAH a dry powder formulation comprising, an active agent, including, treprostinil and a pharmaceutically acceptable carrier and/or excipient, including, a diketopiperazine, wherein the treprostinil in an amount up to 200 μg per dose per dosing session, and the formulation is administered using a dry powder inhaler one or more times daily.

In one embodiment, the dry powder inhaler comprises a movable member for loading a container comprising the pharmaceutical composition and the movable member can configure a container to attain a dosing configuration from a container loading configuration so that the inhaler creates an airflow through the inhaler during an inhalation maneuver to allow the contents of the container to enter the airflow path and greater than 60% of a dry powder dose in the container is delivered to the lungs in a single inhalation. In one embodiment, the method comprises administering a second dry powder composition comprising one or more aforementioned active agents.

In some embodiments, the treatment regimen with an inhalation dry powder depends on the patient's need and can be one inhalation to replace each of a nebulization session performed with standard therapy, including, at least one to four inhalations per day depending on the severity of disease.

### DETAILED DESCRIPTION

In embodiments disclosed herein are methods of treating interstitial lung disease, in particular, pulmonary fibrosis in patients with disease, including, fibrosis of the lungs. In one embodiment, the method comprises administering to a patient in need of treatment one or more dry powder compositions using dry powder inhalers, and delivering the dry powder compositions comprising nintedanib, pirfenidone, and/or treprostinil to the respiratory tract and deep lung.

In an exemplary embodiment a dry powder delivery system comprises a dry powder inhaler for single use of a pharmaceutical dose in a container or a cartridge for delivering the dry powders, including, the pharmaceutical medicaments to a subject by oral inhalation. In one embodiment, the dry powder inhaler is a breath-powered, dry powder inhaler, and the container or cartridge is designed to contain an inhalable dry powder, including, but not limited to pharmaceutical formulations comprising an active ingredient, including a pharmaceutically active substance, and optionally, one or more than one pharmaceutically acceptable carriers and/or excipients. In particular, the dry powder inhaler containing the pharmaceutical compositions are for the treatment of pulmonary fibrosis and/or pulmonary arterial hypertension.

The dry powder inhalers are provided in various embodiments of shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges, and empty cartridges. The present inhalation systems can be designed to be used with any type of dry powder. In one embodiment, the dry powder is a relatively cohesive powder which requires optimal deagglomeration conditions. In one embodiment, the inhalation system provides a re-useable, miniature breath-powered inhaler in combination with single-use cartridges containing pre-metered doses of a dry powder formulation. The inhaler can deliver a dry powder dose in a single inhalation per use in treating interstitial lung disease with or without pulmonary arterial hypertension, in less than 10 seconds, or less than 6 seconds or less than 4 seconds per cartridge session. In particular embodiments, oral inhalation through the inhalers can deliver greater than 60% of a powder dose in less than 6 seconds, in less than 4 seconds and in less than 2 seconds.

As used herein the term “a unit dose inhaler” refers to an inhaler that is adapted to receive a single enclosure, cartridge or container comprising a dry powder formulation and delivers a single dose of a dry powder formulation by inhalation from a single container to a user. It should be understood that in some instances multiple unit doses will be required to provide a user with a specified dosage and that the same inhaler can be used for multiple unit dose delivery and in multiple dose sessions for a predetermined number of use sessions.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa and can attain a closed configuration to hold a dry powder and a dosing configuration in use with an inhaler.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single enclosure including a container having a single dose or multiple doses of formulation that can be delivered by inhalation as metered single amounts. A unit dose enclosure/cartridge/container contains a single dose. Alternatively, it can comprise multiple individually accessible compartments, each containing a unit dose.

As used herein, the term “about” is used to indicate that a value includes the standard deviation of error for the device or method being employed to determine the value.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 μm, though some references use somewhat different ranges, as measured using standard techniques, for example, with an Anderson Cascade Impactor. Other impactors can be used to measure aerodynamic particle size such as the NEXT GENERATION IMPACTOR™ (NGI™, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508,732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of ≥80%, 85%, or 90% and a VMGD of the emitted particles of <12.5 μm, <7.0 μm, or <4.8 μm can indicate progressively better aerodynamic performance.

Respirable fraction on fill (RF/fill) represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. As described herein, a RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

The present disclosure also provides improved powders comprising microcrystalline particles, compositions, methods of making the particles, and therapeutic methods that allow for improved delivery of drugs to the lungs for treating diseases and disorders in a subject and decreases the adverse effects caused by enteral or intravenous therapy. Embodiments disclosed herein achieve improved delivery by providing crystalline diketopiperazine compositions comprising microcrystalline diketopiperazine particles having high capacity for drug adsorption yielding powders having high drug content of one or more active agents. Powders made with the present microcrystalline particles can deliver increased drug content in lesser amounts of powder dose, which can facilitate drug delivery to a patient. The powders can be made by various methods including, methods utilizing surfactant-free solutions or solutions comprising surfactants depending on the starting materials.

In alternate embodiments disclosed herein, the drug delivery system can comprise a dry powder for inhalation comprising a plurality of substantially uniform, microcrystalline particles, wherein the microcrystalline particles can have a substantially hollow spherical structure and comprise a shell which can be porous comprising crystallites of a diketopiperazine that do not self-assemble in a suspension or in solution. In certain embodiments, the microcrystalline particles can be substantially hollow spherical and substantially solid particles comprising crystallites of the diketopiperazine depending on the drug and/or drug content provided and other factors in the process of making the powders. In one embodiment, the microcrystalline particles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by BJH adsorption.

Certain embodiments disclosed herein comprise dry powders comprising, a plurality of substantially uniform, microcrystalline particles, wherein the particles have a substantially spherical structure comprising a shell which can be porous, and the particles comprise crystallites of a diketopiperazine that do not self-assemble in suspension or solution, and have a volumetric median geometric diameter less than 5 μm; or less than 2.5 μm and comprise an active agent.

In a particular embodiment herein, up to about 92% of the microcrystalline particles have a volumetric median geometric diameter of 5.8 μm. In one embodiment, the particle's shell is constructed from interlocking diketopiperazine microcrystals having one or more drugs adsorbed on their surfaces. In some embodiments, the particles can entrap the drug in their interior void volume and/or combinations of the drug adsorbed to the crystallites' surface and drug entrapped in the interior void volume of the spheres.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble; wherein the particles are formed by a method comprising the step of combining diketopiperazine having a trans isomer content ranging from about 45% to 65% in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus. The microcrystalline particles can be pre-formed without for later used, or combined with an active agent in suspension prior to spray drying.

The method can further comprise the steps of, adding with mixing a solution comprising an active agent or an active ingredient such as a drug or bioactive agent along with other pharmaceutically acceptable carriers and/or excipients prior to drying the solution or suspension, for example, prior to the spray drying step. In this manner, the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the microcrystalline particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and without the presence of an active agent, and concurrently homogenizing in a high shear mixer at high pressures of up to 2,000 psi to form a precipitate; washing the precipitate in suspension with deionized water; concentrating the suspension and drying the suspension in a spray drying apparatus.

In certain embodiments wherein the starting material comprising the active ingredient is an extract exhibiting a high degree of viscosity, or a substance having a honey like viscous appearance, the microcrystalline particles are formed as above and by washing them in water using tangential flow filtration prior to combining with the extract or viscous material. After washing in water, the resultant particle suspension is lyophilized to remove the water and re-suspended in an alcohol solution, including ethanol or methanol prior to adding the active ingredient as a solid, or in a suspension, or in solution. In one embodiment, optionally, the method of making the composition comprises the step of adding any additional excipient, including one or more, amino acid, such as leucine, isoleucine, norleucine, methionine or one or more phospholipids, for example, 1,2-dipalmitoyl-sn-glycero-3-phosphocholine (DPPC) or 1,2-distearoyl-sn-glycero-3-phosphocholine (DSPC), concurrently with the active ingredient or subsequent to adding the active ingredient, and prior to spray drying. In certain embodiments, forming the composition comprises the step wherein the extract comprising desired active agents is, optionally, filtered or winterized to separate and remove layers of unwanted materials such as lipids to increase its solubility.

The method can further comprise the steps of adding a solution with mixing to the mixture, and wherein the mixing can optionally be performed with or without homogenization in a high shear mixer, wherein the solution comprises an active agent or an active ingredient such as a drug or bioactive agent prior to the spray drying step so that the active agent or active ingredient is adsorbed and/or entrapped within or on the surface of the particles. Particles made by this process can be in the submicron size range prior to spray-drying, or the particles can be formed from the solution during spray-drying.

In some embodiments herewith, the drug content can be delivered on crystalline powders using FDKP and which are lyophilized or sprayed dried at contents to about 10%, or about 20%, or about 30% or higher. In embodiments using microcrystalline particles formed from FDKP, or FDKP disodium salt, and wherein the particles do not self-assemble and comprise submicron size particles, drug content can typically be greater than 0.01% (w/w). In one embodiment, the drug content to be delivered with the microcrystalline particles of from about 0.01% (w/w) to about 75% (w/w); from about 1% to about 50% (w/w), from about 10% (w/w) to about 25% (w/w), or from about 10% to about 20% (w/w), or from 5% to about 30%, or greater than 25% depending on the drug to be delivered. An example embodiment wherein the drug is a nintedanib, the percent nintedanib or pirfenidone in the composition can comprise from about 1% to about 50% (w/w) of the dry powder content. In certain embodiments, the drug content can be greater in the dry powder composition and can vary depending on the form and size of the drug particles to be delivered.

In an exemplary embodiment, a method of treating interstitial lung disease comprises a dry powder composition comprising microcrystalline particles of fumaryl diketopiperazine, wherein the nintedanib, pirfenidone, or treprostinil is adsorbed to the particles and wherein the content of the treprostinil in the composition comprises up to about 20%, or about 30% (w/w) and ranges from about 0.5% (w/w) to about 20% (w/w) or from about 1% (w/w) to about 10% (w/w), or from about 1% to about 5% (w/w) of the dry powder. In one embodiment, the composition herein can comprise one or more than one excipient suitable for inhalation, including, amino acids, including methionine, histidine, isoleucine and leucine. In this embodiment, for example, a treprostinil, nintedanib, pirfenidone composition can be used in the prevention and treatment of pulmonary fibrosis or pulmonary hypertension and interstitial lung disease by having the patient self-administering an effective dose comprising about 1 mg to 15 mg of a dry powder composition comprising microcrystalline particles of fumaryl diketopiperazine and treprostinil in a single inhalation. In a particular embodiment, the treprostinil content in the formulation can be from about 1 μg to about 200 μg. In one embodiment, the dry powder content of the cartridges comprising treprostinil can be 20 μg, 30 μg, 60 μg, 90 μg, 120 μg, 150 μg, 180 μg, 200 μg, 300 μg, or 500 μg per dose regimen.

In alternate embodiments, the pharmaceutically acceptable carrier for making dry powders can comprise any carriers or excipients useful for making dry powders and which are suitable for pulmonary delivery. Example of pharmaceutically suitable carriers and excipients include, sugars, including saccharides and polysaccharides, such as lactose, mannose, sucrose, mannitol, trehalose; citrates, amino acids such as glycine, L-leucine, isoleucine, trileucine, tartrates, methionine, vitamin A, vitamin E, zinc citrate, sodium citrate, trisodium citrate, sodium tartrate, sodium chloride, zinc chloride, zinc tartrate, polyvinylpyrrolidone, polysorbate 80, phospholipids including diphosphotidylcholine and the like.

In one embodiment, a method of self-administering a dry powder formulation to one's lung(s) with a dry powder inhalation system is also provided. The method comprises: obtaining a dry powder inhaler in a closed position and having a mouthpiece; obtaining a cartridge comprising a pre-metered dose of a dry powder formulation in a containment configuration, wherein the dry powder comprises nintedanib, or pirfenidone, or treprostinil; opening the dry powder inhaler to install the cartridge or capsule; closing the inhaler to effectuate movement of the cartridge to a dose position; placing the mouthpiece in one's mouth, and inhaling once deeply to deliver the dry powder formulation to the lungs in less than 6 seconds.

In another embodiment, a method of treating interstitial lung disease including idiopathic pulmonary fibrosis is disclosed with diketopiperazine-based microparticles as carriers or excipients. The method comprises the administration of an inhalable dry powder composition or formulation comprising, for example, a diketopiperazine having the formula 2,5-diketo-3,6-di(4-X-aminobutyl)piperazine, wherein X is selected from the group consisting of succinyl, glutaryl, maleyl, and fumaryl. In this embodiment, the dry powder composition can comprise a diketopiperazine salt for making amorphous powders. In still yet another embodiment, there is provided a dry powder composition or formulation, wherein 1 the diketopiperazine is 2,5-diketo-3,6-di-(4-fumaryl-aminobutyl)piperazine, with or without a pharmaceutically acceptable carrier, or excipient and the active agent.

An inhalation system for delivering a dry powder formulation to a patient's lung(s) is provided, the system comprises a high resistance dry powder inhaler configured to have flow conduits with a total resistance to flow in a dosing configuration ranging in value from 0.05 to about 0.200 (√kPa)/liter per minute. The dry powder inhaler can be provided comprising a dry powder formulation for single use that can be discarded after use, or with individual doses that are replaceable in a multiple use inhaler and the individual dose enclosures or containers can be discarded after use. Individual dose cartridges comprising the dry powder formulations can be provided in individual packages or multiple cartridge doses can be provided in blister packages.

In one embodiment, a dry powder inhalation kit is provided comprising, a dry powder inhaler as described above, one or more medicament cartridges comprising a dry powder formulation for treating a disorder or disease such as respiratory tract and lung disease, including pulmonary fibrosis, pulmonary arterial hypertension, cystic fibrosis, respiratory infections, cancer, and other systemic diseases, including, endocrine disease, including, diabetes and obesity.

Methods of treating a disease or disorder in a patient with the dry powder inhaler embodiments disclosed herewith is also provided. The method of treatment comprises providing to a patient in need of treatment a dry powder inhaler comprising a cartridge containing a dose of an inhalable formulation comprising an active ingredient selected from the group as described above and a pharmaceutical acceptable carrier and/or excipient; and having the patient inhale through the dry powder inhaler deeply for about 3 to 4 seconds or less than 6 seconds to deliver the dose to the patient's lung. In the method, the patient can resume normal breathing pattern thereafter. Treatment of interstitial lung disease can be sustained for a period of a week, two weeks, three weeks, and up to two months; wherein the administration of for, example, nintedanib to a patient can occur once or twice daily with up to 300 mg, with patient monitoring for any adverse side effects.

In an alternate embodiment, a method is provided for treating disease of the lungs, including, interstitial lung disease, for example, idiopathic pulmonary fibrosis, comprising, administering to a subject in need of treatment an inhalable composition comprising a kinase inhibitor molecule, including, a tyrosine kinase inhibitor and a diketopiperazine of the formula:

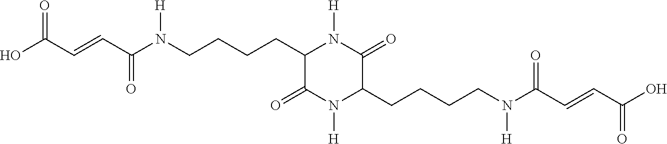 [SMILES: O=C(O)/C=C/C(=O)NCCCCC1NC(=O)C(CCCCNC(=O)/C=C/C(=O)O)NC1=O]

and optionally, one or more pharmaceutical excipients or carriers as define above with respect to the formulation. In one embodiment, the kinase inhibitor molecule, includes, but not limited to axitanib, bosutinib, caboznantinib, crizotinib, dasatinib erlotinib, gefitinib, imatinib, lapatinib, nilotinib, pazopanib, panatinib, regorafenib, ruxolitinib, sorafenib, sunitinib, vandetanib, vemurafenib, and the like.

The following examples illustrate some of the processes for making dry powders suitable for using with the inhalers described herein and data obtained from experiments using the dry powders.

### EXAMPLE 1

### Preparation of Crystalline Composite Nintedanib Dry Powders

A 10% nintedanib solution (concentration of nintedanib in this solution could range from 1% nintedanib to 10% nintedanib) was prepared by adding nintedanib (0.025 g) to a 10% (w/w) acetic acid solution (0.225 g) (concentration of acetic acid solution could range from 10% to 100% acetic acid). The nintedanib solution was added to a microcrystalline particle (XC) suspension (1.31% solids, 188.93 g) suspension of 3,6-bis(N-fumaryl-4-aminobutyl)-2,5-diketopiperazine, or fumaryl diketopiperazine (solids content of the XC suspension could range from 0.5% to 5% (w/w)). The nintedanib XC suspension was spray dried using a Buchi B-290 spray dryer with the conditions shown in Table 1 to produce a 1% (w/w) nintedanib XC powder and yield was about 2.5 g.

TABLE 1
Nintedanib Powder Spray Drying Conditions

| Spray Dryer | | |
|---|---|---|
| Parameter | Set Point | |
| Inlet Temperature | 180° | C. |
| Aspirator Pump Speed | 90% | |
| Feed Pump Speed | 25% | |
| Nitrogen Flow | 60 | m |

### Preparation 20% (w/w) Nintedanib Crystalline XC Powder Preparations

A 10% nintedanib solution (concentration of nintedanib in this solution could range from 1% nintedanib to 10% nintedanib) was prepared by adding nintedanib (3.33 g*) to a 20% acetic acid solution (29.97 g) (concentration of acetic acid solution could range from 10% to 100% acetic acid). Separately, an XC suspension was prepared by adding fumaryl diketopiperazine particles (11.67 g*) to deionized water (705.03 g) (suspension solids=1.63%) (solids content of the XC suspension could range from 0.5% to 5%). The nintedanib solution was then added to the XC suspension and the resulting nintedanib XC suspension was spray dried using a Buchi B-290 spray dryer with the conditions shown in Table 1 to produce a 20% nintedanib XC powder and a resultant yield of about 15 g.

### Preparation of Crystalline Nintedanib T Dry Powders

A 10% nintedanib solution (concentration of nintedanib in this solution could range from 1% nintedanib to 10% nintedanib) was prepared, for example, by adding nintedanib (0.025 g) (nintedanib charge could range from 0.025 g to 050 g) to a 10% acetic acid solution (0.225 g) (concentration of acetic acid solution could range from 10% to 100% acetic acid). The nintedanib solution was added to a suspension of 3,6-bis(N-fumaryl-4-aminobutyl)-2,5-diketopiperazine pre-formed particles (T suspension; 8.11% solids, 30.52 g) (solids content of the T suspension could range from 0.5% to 20% (w/w)) as described below. The nintedanib T suspensions were then dried either by spray drying or by lyophilization to produce 1% nintedanib T powders. Spray dried powders were dried using a Buchi B-290 spray dryer with conditions shown in Table 1. Lyophilized powders were prepared by first pelletizing the nintedanib T suspension into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and the resultant yield was about 2.5 g.

### Preparation of Spray Dried 20% (w/w) Nintedanib Crystalline T Powder

A 10% nintedanib solution (concentration of nintedanib in this solution could range from 1% nintedanib to 10% nintedanib) was prepared by adding nintedanib (3.33 g*) to a 20% acetic acid solution (30.0 g) (concentration of acetic acid solution could range from 10% to 100% acetic acid). The nintedanib solution was added to a T suspension (8.99% solids, 129.81 g) (solids content of the T suspension could range from 0.5% to 20. The nintedanib T suspension was then spray dried using a Buchi B-290 spray dryer with conditions shown in Table 1. The resultant yield was about 15 g.

### Preparation of Lyophilized 20% Nintedanib T Powder

A 10% nintedanib solution (concentration of nintedanib in this solution could range from 1% nintedanib to 10% nintedanib) was prepared by adding nintedanib (2.63 g*) to a 10% acetic acid solution (23.63 g) (concentration of acetic acid solution could range from 10% to 100% acetic acid). The nintedanib solution was added to a T suspension (8.99% solids, 104.23 g*) (solids content of the T suspension could range from 0.5% to 20. The nintedanib T suspension was lyophilized by first pelletizing the nintedanib T suspension into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and resulted in about 12 g yield.

### Preparation of Lyophilized 20% Nintedanib T Powder with Reduced Solids Content

A 10% nintedanib solution (concentration of nintedanib in this solution could range from 1% nintedanib to 10% nintedanib) was prepared by adding nintedanib (3.09 g*) to a 20% acetic acid solution (27.81 g) (concentration of acetic acid solution could range from 10% to 100% acetic acid). Separately, the T suspension (8.99% solids, 132.48 g*) was diluted with deionized water (136.62 g) (solids content of the T suspension could range from 0.5% to 20% (w/w). The nintedanib solution was added to this diluted T suspension resulting in a nintedanib T suspension with a solids content of 5.00%. The nintedanib T suspension was lyophilized by first pelletizing it into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and the resultant yield was about 15 g.

### Preparation of Lyophilized 20% Nintedanib T Powder with Reversed Component Addition

A 10% nintedanib solution (concentration of nintedanib in this solution could range from 1% nintedanib to 10% nintedanib) was prepared by adding nintedanib (3.09 g*) to a 20% acetic acid solution (27.81 g) (concentration of acetic acid solution could range from 10% to 100% acetic acid). The nintedanib solution was diluted with deionized water (97.19 g). Lyophilized T particles (11.91 g*) were then added to the nintedanib solution, portionwise, over 4 min. Deionized water (10.00 g) was used to wash the residual lyophilized T particles into the nintedanib T suspension. The nintedanib T suspension was lyophilized by first pelletizing it into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and resultant yield was about 15 g.

### Preparation of Lyophilized 20% Nintedanib Esylate T Powder

A 1% nintedanib esylate solution (concentration of nintedanib esylate in this solution could range from 1% nintedanib esylate to 5% nintedanib esylate) was prepared by adding nintedanib esylate (3.61 g*) portionwise to deionized water (357.39 g). The nintedanib esylate solution was added to the T suspension (8.99% solids, 126.70 g*) (solids content of the T suspension could range from 0.5% to 20%) and the resulting nintedanib esylate T suspension was pelletized into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and resultant yield was about 15 g.

### Powder Testing

Powders were evaluated for geometric particle size distribution using a Sympatec laser diffraction instrument fitted with a RODOS bulk powder dispersing system. Bulk powders were dispersed at 0.5 bar and 3.0 bar. Powders were also evaluated for aerodynamic particle size distribution using an Andersen Cascade impactor (ACI). Powders were discharged through the ACI from Gen 2C cartridges (10 mg cartridge fills) at 4 kPa. Data for the nintedanib powders are shown in Table 2.

Table 2. Nintedanib Powder Data

TABLE 2
Nintedanib Powder Data

| | | | | | RODOS DATA | | | | ACI Data | |
|---|---|---|---|---|---|---|---|---|---|---|
| | | % | | Ninte. | 0.5 bar | | 3.0 bar | | Avg. | |
| Powder | Yield | Solid | Yield | Assay | ×(50) | ×(90) | ×(50) | ×(90) | CE | RF |
| Type | (g) | (%) | (%) | (Wt %) | (μm) | (μm) | (μm) | (μm) | (%) | (%) |

XC Powders

| | | | | | | | | | | |
|---|---|---|---|---|---|---|---|---|---|---|
| 1% Nintedanib | 2.5 | 1.32 | 78.4 | 1.01 | 4.34 | 8.54 | 3.14 | 7.94 | 89.1 | 27.9 |
| XC Powder | | | | | | | | | | |
| 1% Nintedanib | 2.5 | 1.32 | 77.6 | 0.95 | 4.19 | 9.12 | 3.33 | 10.09 | 75.7 | 25.0 |
| XC Powder | | | | | | | | | | |
| 10% Nintedanib | 2.5 | 2.06 | 80.0 | 8.52 | 2.19 | 5.00 | 1.73 | 4.30 | 95.2 | 45.4 |
| XC Powder | | | | | | | | | | |
| 20% Nintedanib | 2.5 | 2.26 | 82.8 | 17.94 | 2.41 | 5.55 | 1.89 | 5.21 | 90.9 | 46.1 |
| XC Powder | | | | | | | | | | |
| 20% Nintedanib | 15.0 | 2.00 | 67.0 | 20.70 | 2.14 | 5.82 | 1.51 | 3.69 | 83.9 | 41.7 |
| XC Powder | | | | | | | | | | |

Spray Dried T Powders

| | | | | | | | | | | |
|---|---|---|---|---|---|---|---|---|---|---|
| 1% Nintedanib | 2.5 | 8.12 | 80.0 | 1.08 | 1.54 | 3.60 | 1.31 | 2.44 | 91.8 | 72.2 |
| T Powder | | | | | | | | | | |
| 1% Nintedanib | 2.5 | 8.12 | 79.2 | 0.95 | 1.50 | 3.34 | 1.35 | 2.65 | 79.7 | 53.8 |
| T Powder | | | | | | | | | | |
| 10% Nintedanib | 2.5 | 7.46 | 82.8 | 8.37 | 1.74 | 3.81 | 1.42 | 2.73 | 76.2 | 43.7 |
| T Powder | | | | | | | | | | |
| 20% Nintedanib | 2.5 | 7.68 | 82.8 | 18.07 | 1.94 | 4.33 | 1.52 | 2.87 | 87.5 | 36.7 |
| T Powder | | | | | | | | | | |
| 20% Nintedanib | 15 | 9.19 | 88.5 | 20.30 | 2.01 | 4.32 | 1.41 | 2.79 | 78.6 | 48.6 |
| T Powder | | | | | | | | | | |

Lyophilized T Powders

| | | | | | | | | | | |
|---|---|---|---|---|---|---|---|---|---|---|
| 1% Nintedanib | 2.5 | 8.12 | 97.2 | 1.15 | 1.75 | 4.58 | 1.33 | 2.48 | 97.2 | 60.6 |
| T Powder | | | | | | | | | | |
| 1% Nintedanib | 2.5 | 8.12 | 97.6 | 0.88 | 2.09 | 5.05 | 1.58 | 3.13 | 92.1 | 54.0 |
| T Powder | | | | | | | | | | |
| 10% Nintedanib | 2.5 | 7.46 | 95.6 | 9.27 | 1.98 | 4.27 | 1.48 | 2.84 | 62 | 38.2 |
| T Powder | | | | | | | | | | |
| 20% Nintedanib | 2.5 | 7.68 | 94.4 | 18.28 | 2.45 | 5.41 | 1.71 | 3.28 | 88 | 45.1 |
| T Powder | | | | | | | | | | |
| 20% Nintedanib | 12 | 9.20 | 94.4 | 21.30 | 3.78 | 9.05 | 1.89 | 4.31 | 87.3 | 32.2 |
| T Powder | | | | | | | | | | |
| 20% Nintedanib | 15 | 5.00 | 98.8 | 20.72 | 2.76 | 6.28 | 1.95 | 3.94 | 83.9 | 36.4 |
| T Powder | | | | | | | | | | |
| 20% Nintedanib | 15 | 10.00 | 97.6 | 20.19 | 3.09 | 7.72 | 2.02 | 4.17 | 83.3 | 33.4 |
| T Powder- | | | | | | | | | | |
| Reverse | | | | | | | | | | |
| Addition | | | | | | | | | | |
| 20% Nintedanib | 15 | 3.08 | 100.9 | 18.99 | 3.76 | 9.76 | 2.13 | 5.09 | 50.2 | 6.1 |
| Esylate T | | | | | | | | | | |
| Powder | | | | | | | | | | |

As can be seen in Table 2, the process product yield was greater than about 67% in the composition reactions for both sprayed dried or lyophilized dry powders. In addition, it can be seen that the percent yield was improved for the lyophiplized T powders, and all powders containing 10 wt % and 20 wt % nintedanib in the composition, no matter the method of making the powders. The data show the average powder delivered from the delivery system was greater than 75% for all the XC powder and spray-dried T powders, and greater than or equal to 62% for all lyophilized T powders, as assessed by cartridge emptying (CE) measurements with some powders yielding upwards of about 97% CE. The data also illustrates that the XC powders at higher concentration (10 wt % and 20wt %) appear to have consistent cartridge emptying performance than at lower concentrations, however the best CE performance powders were the 1% T powders either sprayed dried or lyophilized.

### EXAMPLE 2

### Preparation of Crystalline Composite Pirfenidone Dry Powders

A 25% pirfenidone solution (concentration of pirfenidone in this solution can range from 1% pirfenidone to 40% pirfenidone) was prepared by adding pirfenidone (0.20 g) (the pirfenidone charge used to prepare these powders was varied between 0.2 g and 0.63 g) to ethanol (0.60 g) (when a 25% pirfenidone solution was used, water could be added to the ethanol up to a 50:50 weight ratio of ethanol:water). The pirfenidone solution was added to an microcrystalline (XC) suspension (1.31% solids, 137.40 g) (solids content of the XC suspension could range from 0.5% to 5%). The pirfenidone XC suspension was spray dried using a Buchi B-290 spray dryer with the conditions shown in Table 1 to produce a pirfenidone XC powder.

### Preparation of Crystalline Pirfenidone Powders

A 25% pirfenidone solution (concentration of pirfenidone in this solution could range from 1% pirfenidone to 40% pirfenidone) was prepared by adding pirfenidone (0.20 g) (the pirfenidone charge used to prepare these powders was varied between 0.2 g and 0.33 g) to ethanol (0.60 g) (when a 25% pirfenidone solution was used, water could be added to the ethanol up to a 50:50 weight ratio of ethanol:water). The pirfenidone solution was added to a T suspension (8.11% solids, 22.19 g) (solids content of the T suspension could range from 0.5% to 20%). The pirfenidone T suspensions were then dried either by spray drying or by lyophilization to produce pirfenidone T powders. Spray dried powders were dried using a Buchi B-290 spray dryer with conditions shown in Table 1. Lyophilized powders were prepared by first pelletizing the pirfenidone T suspension into liquid nitrogen followed by drying in a Virtis Genesis 25 XL shelf lyophilizer. The lyophilizer was run on a program where the shelf temperature was ramped from −45° C. to 25° C. at 0.2° C./min and maintained at 25° C. under vacuum until the powder was completely dried.

### Preparation of Amorphous Pirfenidone Powders

A 25% pirfenidone solution (concentration of pirfenidone in this solution could range from 40% pirfenidone to 1% pirfenidone) was prepared by adding pirfenidone (0.20 g) (the pirfenidone charge used to prepare these powders was varied between 0.2 g and 0.22 g) to ethanol (0.60 g) (when a 25% pirfenidone solution was used, water could be added to the ethanol up to a 50:50 weight ratio of ethanol:water). Separately a 10% FDKP-disodium salt solution was prepared. Leucine (leucine charge ranged from 0 g to 0.45 g), and FDKP-disodium salt (1.80 g) were dissolved in in deionized water (16.20 g) (concentration of the FDKP-disodium salt in this solution could range from 5% to 20%). The pirfenidone solution was added to the FDKP-disodium salt solution and the resulting solution was spray dried using a Buchi B-290 spray dryer run using the conditions shown in Table 1 to produce pirfenidone amorphous powders.

The preceding disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments disclosed herein are illustrative of the principles of the present invention. Other modifications that may be employed are within the scope of the invention. Thus, by way of example, but not of limitation, alternative configurations may be utilized in accordance with the teachings herein. Accordingly, the present invention is not limited to that precisely as shown and described.

We claim:

1. A method of treatment comprising, administering to a patient in need of treatment for interstitial lung disease by inhalation a therapeutically effective amount of a dry powder pharmaceutical composition comprising one or more active agents and a diketopiperazine.

2. The method of claim 1, wherein the one or more active agents is a vasodilator.

3. The method of claim 2, wherein the vasodilator is a prostaglandin, a prostaglandin derivative, a prostaglandin analog, a salt thereof, or a prodrug thereof.

4. The method of claim 3, wherein the prostaglandin analog is prostacyclin or an analog thereof.

5. The method of claim 1, wherein the one or more active agents is selected from sildenafil, tadalafil, and vardenafil.

6. The method of claim 1, wherein the one or more active agents are nintedanib: 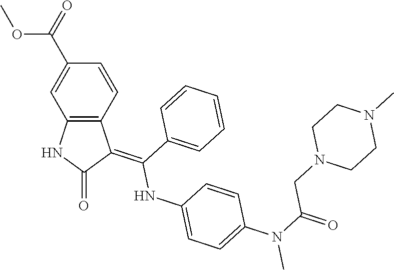
and one of a prostaglandin, a prostaglandin derivative, a prostaglandin analog, a salt thereof or a prodrug thereof, wherein the nintedanib is administered to the patient in a dry powder composition separately.

7. The method of claim 4, wherein the prostaglandin analog is treprostinil, or a Treprostinil salt thereof, or treprostinil ester thereof.

8. The method of claim 1, wherein the diketopiperazine is provided as microcrystalline particles of 3,6-bis(N-fumaryl-4-aminobutyl)-2,5-diketopiperazine.

9. The method of claim 8, wherein the microcrystalline particles have a specific surface area ranging from about 20 m²/g to about 63 m²/g.

10. The method of claim 6, wherein the nintedanib is provided to the patient in doses of 1 mg to about 10 mg, 10 mg to 20 mg, or 20 mg to 30 mg per inhalation at least once per day.

11. The method of claim 1, wherein the interstitial lung disease is idiopathic pulmonary fibrosis.

12. The method of claim 1, wherein the patient is diagnosed with pulmonary hypertension and interstitial lung disease.

13. The method of claim 1, wherein the dry powder pharmaceutical formulation is for oral inhalation using a dry powder inhaler.

14. The method of claim 6, wherein the prostaglandin analog is prostacyclin, prostacyclin analog, or a salt thereof.

15. A method of treatment comprising, administering to a patient in need of treatment for interstitial lung disease a combination therapy comprising a dose of nintedanib in a dry powder pharmaceutical composition for oral inhalation, or a dose of treprostinil, wherein the dose of nintedanib and treprostinil are administered in separate cartridges using a dry powder inhaler.

16. The method of claim 15, wherein the dose of Treprostinil is in an amount of 10 μg to 300 μg per dose per dosing session one or more times per day.

17. The method of claim 15, wherein a dose of nintedanib is up to 300 mg per day administered in once or more than once times per day.

18. The method of claim 15, wherein the interstitial lung disease is idiopathic pulmonary fibrosis.

19. The method of claim 15, wherein the nintedanib is administered in an amount of up to 30 mg of an inhalable dry powder per cartridge.

20. The method of claim 15, wherein the nintedanib is nintedanib esylate.

21. A dry powder pharmaceutical composition comprising one or more active agents and a diketopiperazine, wherein the one or more active agents is selected from sildenafil, tadalafil, and vardenafil.

22. A dry powder pharmaceutical composition comprising one or more active agents and a diketopiperazine, wherein the one or more active agents is selected from a prostaglandin, a prostaglandin derivative, a prostaglandin analog, a salt thereof, and a prodrug thereof.
